(19) United States
(12) Patent Application Publication
(10) Pub. No.: US 2010/0305655 A1
(43) Pub. Date: Dec. 2, 2010

(54) METHODS AND APPARATUS FOR TREATING GASTROINTESTINAL DISORDERS USING ELECTRICAL SIGNALS

(75) Inventors: John T. Raffle, Austin, TX (US); Joseph P. Errico, Green Brook, NJ (US); Bruce Simon, Mountain Lakes, NJ (US)

Correspondence Address: ELECTROCORE INC., 51 GILBRALTAR DRIVE, SUITE 2F, POWER MILL PLAZA, MORRIS PLAINS, NJ, 07950-1254, US

(73) Assignee: ElectroCore, LLC, Morris Plains, NJ (US)

(21) Appl. No.: 12/787,583

(22) Filed: May 26, 2010

Related U.S. Application Data
(60) Provisional application No. 61/181,820, filed on May 28, 2009.

(51) Int. Cl. A61N 1/36

(52) U.S. Cl. 607/40

(57) ABSTRACT

A method of treating motility conditions, such as an arrest of intestinal peristalsis, includes introducing an electrode through the esophagus and into the digestive tract of the patient and applying an electrical impulse to the electrode to modulate one or more nerves within the digestive tract such that intestinal peristalsis function is at least partially improved.

### CROSS REFERENCE TO RELATED APPLICATIONS

This application claims the benefit of U.S. Provisional Patent Application No. 61/181,820 filed May 28, 2009; the complete disclosures of which are incorporated herein by reference for all purposes. This application is related to U.S. Provisional Patent Application Nos.: 60/792,823 and 60/978,240, the entire disclosures of which are hereby incorporated by reference. This application is also related to commonly assigned co-pending U.S. patent Ser. Nos. 12/246,605, 11/735,709, 11/555,142, 11/555,170, 11/592,095, 11/591,340, 11/591,768 and 11/754,522, the complete disclosures of which are incorporated herein by reference for all purposes.

### BACKGROUND OF THE INVENTION

The present invention relates to the field of delivery of electrical impulses to bodily tissues for therapeutic purposes, and more specifically to devices and methods for treating gastrointestinal disorders or diseases, such as conditions associated with an arrest of intestinal peristalsis, e.g., paralytic Ileus, adynamic Ileus, and/or paresis.

The use of electrical stimulation for treatment of medical conditions has been well known in the art for nearly two thousand years. One of the most successful modern applications of this basic understanding of the relationship between muscle and nerves is the cardiac pacemaker. Although its roots extend back into the 1800's, it wasn't until 1950 that the first practical, albeit external and bulky pacemaker was developed. Dr. Rune Elqvist developed the first truly functional, wearable pacemaker in 1957. Shortly thereafter, in 1960, the first fully implanted pacemaker was developed. Around this time, it was also found that the electrical leads could be connected to the heart through veins, which eliminated the need to open the chest cavity and attach the lead to the heart wall. In 1975 the introduction of the lithium-iodide battery prolonged the battery life of a pacemaker from a few months to more than a decade. The modern pacemaker can treat a variety of different signaling pathologies in the cardiac muscle, and can serve as a defibrillator as well (see U.S. Pat. No. 6,738,667 to Deno, et al., the disclosure of which is incorporated herein by reference).

There are two types of intestinal obstructions, mechanical and non-mechanical. Mechanical obstructions occur because the bowel is physically blocked and its contents can not pass the point of the obstruction. This happens when the bowel twists on itself (volvulus) or as the result of hernias, impacted feces, abnormal tissue growth, or the presence of foreign bodies in the intestines. Ileus is a partial or complete non-mechanical blockage of the small and/or large intestine. Unlike mechanical obstruction, non-mechanical obstruction, Ileus or paralytic Ileus, occurs because peristalsis stops. Peristalsis is the rhythmic contraction that moves material through the bowel.

Ileus may be associated with an infection of the membrane lining the abdomen, such as intraperitoneal or retroperitoneal infection, which is one of the major causes of bowel obstruction in infants and children. Ileus may be produced by mesenteric ischemia, by arterial or venous injury, by retroperitoneal or intra-abdominal hematomas, after intra-abdominal surgery, in association with renal or thoracic disease, or by metabolic disturbances (e.g., hypokalemia).

Gastric and colonic motility disturbances after abdominal surgery are largely a result of abdominal manipulation. The small bowel is largely unaffected, and motility and absorption are normal within a few hours after operation. Stomach emptying is usually impaired for about twenty four hours, but the colon may remain inert for about forty-eight to seventy-two hours (and in some cases 4-7 days). These findings may be confirmed by daily plain x-rays of the abdomen taken postoperatively; they show gas accumulating in the colon but not in the small bowel. Activity tends to return to the cecum before it returns to the sigmoid. Accumulation of gas in the small bowel implies that a complication (e.g., obstruction, peritonitis) has developed.

Symptoms and signs of Ileus include abdominal distention, vomiting, constipation, and cramps. Auscultation usually reveals a silent abdomen or minimal peristalsis. X-rays may show gaseous distention of isolated segments of both small and large bowel. At times, the major distention may be in the colon. When a doctor listens with a stethoscope to the abdomen there will be few or no bowel sounds, indicating that the intestine has stopped functioning. Ileus can be confirmed by x rays of the abdomen, computed tomography scans (CT scans), or ultrasound. It may be necessary to do more invasive tests, such as a barium enema or upper GI series, if the obstruction is mechanical. Blood tests also are useful in diagnosing paralytic Ileus.

Conventionally, patients may be treated with supervised bed rest in a hospital, and bowel rest—where nothing is taken by mouth and patients are fed intravenously or through the use of a nasogastric tube. In some cases, continuous nasogastric suction may be employed, in which a tube inserted through the nose, down the throat, and into the stomach. A similar tube can be inserted in the intestine. The contents are then suctioned out. In some cases, especially where there is a mechanical obstruction, surgery may be necessary. Intravenous fluids and electrolytes may be administered, and a minimal amount of sedatives. An adequate serum K level (>4 mEq/L [>4 mmol/L]) is usually important. Sometimes colonic Ileus can be relieved by colonoscopic decompression. Cecostomy is rarely required.

Drug therapies that promote intestinal motility (ability of the intestine to move spontaneously), such as cisapride and vasopressin (Pitressin), are sometimes prescribed. Some reported opiate therapies (such as alvimopan, a μ-opiod antagonist) are directed to inhibiting the deleterious effects of opioids, to improve intestinal peristalsis.

Alternative practitioners offer few treatment suggestions, but focus on prevention by keeping the bowels healthy through eating a good diet, high in fiber and low in fat. If the case is not a medical emergency, homeopathic treatment and traditional Chinese medicine can recommend therapies that may help to reinstate peristalsis.

Ileus persisting for more than about one week usually involves a mechanical obstructive cause, and laparotomy is usually considered. Colonoscopic decompression may be helpful in cases of pseudo-obstruction (Ogilvie's syndrome), which consists of apparent obstruction at the splenic flexure, although no associated cause is found by barium enema or colonoscopy for the failure of gas and feces to pass.

Post-operative ileus (POI) is a common transient bowel dysmotility. POI is a frequent complication seen in a preponderance of major abdominal surgeries, as well as one of the most frequently encountered sequela of intra-peritoneal chemotherapy. The signs and symptoms associated with POI include abdominal pain and distension, reduced borborygmi, vomiting, nausea, early satiety, and an increased transit time for the passage of flatus and/or stool. POI frequently results in prolonged hospital stays as a consequence of gastrointestinal (GI) complications. Recent estimates of the medical costs incurred due to these complications exceed $1 billion annually. Clinical complications associated with POI include an increase in nasogastric tube reinsertion, intravenous volume maintenance and/or hydration, added nursing care, additional laboratory testing, increased re-admission, and more days in-hospital.

Unfortunately, many lengthy post operative stays in the hospital are associated with Ileus, where the patient simply cannot be discharged until his bowels move. The clinical consequences of postoperative Ileus can be profound. Patients with Ileus are immobilized, have discomfort and pain, and are at increased risk for pulmonary complications. Ileus also enhances catabolism because of poor nutrition. It has been reported in the 1990's that, overall, Ileus prolongs hospital stays, costing $750 million annually in the United States. Thus, it stands to reason that the healthcare costs associated with Ileus over a decade later are much higher. The relatively high medical costs associated with such post operative hospital stays are clearly undesirable, not to mention patient discomfort, and other complications. There are not, however, any commercially available medical equipment that can treat Ileus. It is therefore desirable to avoid the complications associated with the temporary arrest of intestinal peristalsis, particularly that resulting from abdominal surgery, and provide equipment capable of delivering an internal or external treatment to reduce and/or eliminate the pathological responses that are associated with Ileus.

### SUMMARY OF THE INVENTION

The present invention includes systems, devices and methods for treating gastrointestinal disorders or diseases such as the arrest of intestinal peristalsis, dysphagia, gastroesophageal reflux diseases, functional dyspepsia, gastroparesis, irritable bowel syndrome, constipation, diarrhea, fecal incontinence, obesity and eating disorders. Specifically, the method of the present invention includes introducing an electrode device into the digestive tract of a patient and applying an electrical impulse to the electrode to treat the disorder or disease.

In one aspect of the invention, a method for treating the partial or complete arrest of intestinal peristalsis includes introducing an electrode device through the esophagus and into the digestive tract of a patient and applying an electrical impulse to the electrode device to modulate one or more nerves within the digestive tract such that the intestinal peristalsis function is at least partially improved. The electrical impulse is preferably sufficient to initiate local peristalsis in the region immediately surrounding the electrode.

In one embodiment, the introducing step is carried out by positioning the electrode device within the patient's mouth such that the electrode device can be swallowed by the patient (e.g., an electrode “pill”). In another embodiment, the electrode device is manually advanced through the esophagus and into the small intestines of the patient with an introducing device such as a cannula, wire, scope or the like. The electrode device may be introduced to the esophagus through the patient's mouth or nose.

In another aspect of the invention, the method comprises introducing the electrode device through the rectum of the patient and into the digestive tract. In this embodiment, the electrode device is advanced in the opposite direction relative to the natural flow of the digestive tract.

In preferred embodiments, the electrode device is advanced through the digestive tract until it reaches a target region wherein peristalsis is decreased below normal or entirely arrested. An electrical impulse is then applied to the electrode until local peristalsis increased or returns and then the electrode is advanced further down the digestive tract to a second target region wherein peristalsis is hindered or arrested. In this manner, the electrode can be advanced throughout the entire digestive tract, reversing paralytic ileus along its path. Finally, the electrode will reach the large intestine, where it is excreted by the patient or manually removed by the physician.

In one embodiment, the electrode advances naturally through peristalsis until it reaches the region wherein peristalsis is arrested. In other embodiments, the electrode is advanced through external or internal devices, such as external magnets or a wire or cable attached to the electrode. In these latter embodiments, the electrode can be held in place until an appropriate amount and level of electrical impulse(s) has been applied to the target region.

In certain embodiments, the method of the present invention is particularly useful in treating post-operative ileus. In these embodiments, the electrode may be placed at the time of surgery and then activated immediately or after an appropriate period of time. Alternatively, the electrode can be swallowed or placed with a scope when signs of prolonged ileus appear a few days after surgery. The outside of the electrode is preferable smooth and flexible to allow easy passage through the bowels, especially past the surgical site which is held together with sutures.

The electrode device is preferably capsule shaped although other shapes can be used provided that the electrode device is sized and shaped for transport through the gastrointestinal tract. The electrode device comprises a biocompatible, non-digestible material, such as a biocompatible metal or plastic. Alternatively, the electrode may be coasted with, or housed within, a biocompatible, non-digestible material.

In one embodiment, the electrode comprises both a cathode and an anode, preferably positioned on either side of the electrode. In another embodiment, the surface of the electrode device can have a checker board pattern of alternating anodes and cathodes which would then deliver the appropriate signal wherever opposite polarity electrodes came in contact the walls of the intestines. In yet another embodiment, the electrode device acts solely as either the anode or cathode (e.g. the whole pill is the electrode) and the return electrode trails behind it by a small wire attachment. In this embodiment, the entire electrode device may be electrified so that it does not matter which portion of the electrode device contacts the intestine. In addition, it may allow for directional polarization of the intestines, if necessary, to cause peristalsis to proceed in the appropriate direction, i.e. toward the anus. The return electrode or anode is preferably housed in a thin, porous covering to prevent accidental direct contact with the pill electrode, preventing short circuiting and to prevent anodic contact with the walls of the intestine. Alternatively, the return electrode may be a return pad positioned on the exterior skin surface of the patient.

In other embodiments, the device may comprise an electrode array and a tilt sensor. In this embodiment, electrical impulses will automatically switch to different electrodes within the array based on the orientation of the electrode device to maintain the correct orientation of the electric field as the electrode pill passes through the patient's GI tract.

In yet another embodiment, the device includes an external magnet designed to assist with the advancement of the electrode through the patient's GI tract. In addition, the magnet can be used to orient the electrode device properly within the intestines prior to applying the electrical impulse. In preferred embodiments, the magnet can be configured to enhance imaging of the electrode device to allow the physician to view the location of the electrode device and diagnose the patient's ailment. In addition, the magnet may be used to hold the electrode device in place while the signal is being applied such that the electrical impulse can be maintained at the target region along the intestines long enough to ensure that the segment was back to functioning normally.

In yet another embodiment, the device includes a long thin cable comprising a series of nodes or electrodes that are configured to pulse in sequence to pace the stomach and/or intestines into proper function. The cable is advanced through the esophagus and into the target position within the stomach and/or intestines. The cable has a width of about 1-4 mm, preferably about 2.5 mm and the nodes and electrodes are about 2-7 mm wide, preferably about 5 mm and about 0.2 to 3 cm long, preferably about 1 cm. The cable may be driven externally until through the gastrointestinal tract until its distal end has reached the rectum. At this point, the proximal end is disconnected from the signal generator and the device is pulled free.

In yet another embodiment, the device can be further provided with a sensor system for detecting environmental conditions around the housing. In one embodiment, the sensor system includes a movement sensor for detecting peristalsis-induced movement at the region of the electrode device. The sensor system further includes a feedback control system that causes the electrode device to apply electrical impulses only when the peristalsis-induced movement is below a certain threshold velocity or if there is no movement at all. As discussed above, the electrical impulses are designed specifically to stimulate or restart peristalsis in the local region around the electrode device. In other embodiments, the sensor system determines the location of the device within the gastrointestinal tract of the user. In addition or alternatively, the sensor system can include one or more sensors for sensing a variety of different types of environmental factors which may surround the electrode device. For example, mechanical contractions, pressure, temperature, pH or the like.

The electric current, electric field and/or electromagnetic field may be directly applied to the muscles within the GI tract (e.g., within the stomach, small or large intestines) to directly stimulate those muscles and generate peristalsis. Alternatively, the electrical impulse(s) may be applied to nerves that are responsible for peristalsis, such as the enteric nerve system or the parasympathetic or sympathetic nerve chains.

The protocol of one or more embodiments of the present invention may include measuring a response of the patient to the applied current and/or field(s). For example, the digestive muscle activity of the patient may be monitored and the parameters of the drive signal (and thus the induced current and/or fields) may be adjusted to improve the treatment.

Other aspects, features, and advantages of the present invention will be apparent to one skilled in the art from the description herein taken in conjunction with the accompanying drawings.

### DESCRIPTION OF THE DRAWINGS

For the purposes of illustration, there are forms shown in the drawings that are presently preferred, it being understood, however, that the invention is not limited to the precise arrangements and instrumentalities shown.

FIG. 1 illustrates an exemplary electrode device according to the present invention;

FIG. 2 illustrates an alternative embodiment of an electrode device;

FIG. 3 illustrates yet another alternative embodiment of an electrode device in use within the intestines of a patient;

FIG. 4 illustrates another embodiment of the present invention wherein the electrode device comprises a long wire with multiple electrodes;

FIG. 5 is a schematic diagram of an apparatus for electrically stimulating, blocking and/or modulating the nerve fibers within a patient's GI tract; and

FIG. 6 is a graphical illustration of an electrical signal profile that may be used to treat disorders through neuromuscular modulation in accordance with one or more embodiments of the present invention.

### DETAILED DESCRIPTION OF THE PREFERRED EMBODIMENTS

In the present invention, electrical energy is applied to a target region within a patient's body. The invention is particularly useful for applying electrical impulses that interact with the signals of one or more nerves, or muscles, to achieve a therapeutic result, such as the treatment of gastrointestinal disorders. Specifically, the method of the present invention includes introducing an electrode device into the digestive tract of a patient and applying an electrical impulse to the electrode to treat the disorder or disease. For convenience, the remaining disclosure will be directed specifically to the treatment of the temporary arrest of intestinal peristalsis associated with post-operative ileus, but it will be appreciated that the systems and methods of the present invention can be applied equally well to other gastrointestinal disorders, such as dysphagia, gastroesophageal reflux diseases, functional dyspepsia, gastroparesis, irritable bowel syndrome, increasing or decreasing gastric emptying rates, constipation, nausea, vomiting, IBS, diarrhea, fecal incontinence, obesity and eating disorders.

Ileus occurs from hypomotility of the gastrointestinal tract in the absence of a mechanical bowel obstruction. This suggests that the muscle of the bowel wall is transiently impaired and fails to transport intestinal contents. This lack of coordinated propulsive action leads to the accumulation of both gas and fluids within the bowel. Although Ileus has numerous causes, the postoperative state is the most common scenario for Ileus development. Frequently, Ileus occurs after intraperitoneal operations, but it may also occur after retroperitoneal and extra-abdominal surgery. The longest duration of Ileus has been reported to occur after colonic surgery.

According to some hypotheses, postoperative Ileus is mediated via activation of inhibitory spinal reflex arcs. Anatomically, three distinct reflexes are involved: ultrashort reflexes confined to the bowel wall, short reflexes involving prevertebral ganglia, and long reflexes involving the spinal cord. Spinal anesthesia, abdominal sympathectomy, and nerve-cutting techniques have been demonstrated to either prevent or attenuate the development of Ileus. The surgical stress response leads to systemic generation of endocrine and inflammatory mediators that also promote the development of Ileus. Rat models have shown that laparotomy, eventration, and bowel compression lead to increased numbers of macrophages, monocytes, dendritic cells, T cells, natural killer cells, and mast cells, as demonstrated by immunohistochemistry. Calcitonin gene-related peptide, nitric oxide, vasoactive intestinal peptide, and substance P function as inhibitory neurotransmitters in the bowel nervous system. Nitric oxide and vasoactive intestinal peptide inhibitors and substance P receptor antagonists have been demonstrated to improve gastrointestinal function.

In accordance with one or more embodiments of the present invention, a method of treating an arrest of intestinal peristalsis (such as Ileus) includes inducing an electric current, an electric field and/or an electromagnetic field in the GI tract of a patient. The electric current, electric field and/or electromagnetic field may be induced by way of externally disposed apparatus, such as a control unit (including a drive signal generator) or entirely subcutaneous components, including the control unit and/or signal generator.

A method for treating the partial or complete arrest of intestinal peristalsis includes introducing an electrode device through the esophagus and into the digestive tract of a patient and applying an electrical impulse to the electrode device to modulate one or more nerves within the digestive tract such that the intestinal peristalsis function is at least partially improved. The electrical impulse is sufficient to initiate local peristalsis in the region immediately surrounding the electrode. In one embodiment, the introducing step is carried out by positioning the electrode device within the patient's mouth such that the electrode device can be swallowed by the patient (e.g., an electrode “pill”). In another embodiment, the electrode device is manually advanced through the esophagus and into the small intestines of the patient with an introducing device such as a cannula, wire, scope or the like. The electrode device may be introduced to the esophagus through the patient's mouth or nose.

The electrode device may be completely autonomous and self-contained, i.e., where all of the electrical components are completely or substantially contained within a housing or shell and where the device does not require any wires or cables to receive power. For example, power may be provided by an internal battery or wireless receiving system. Alternatively, an external power source may be used that provides power remotely to the electrode device.

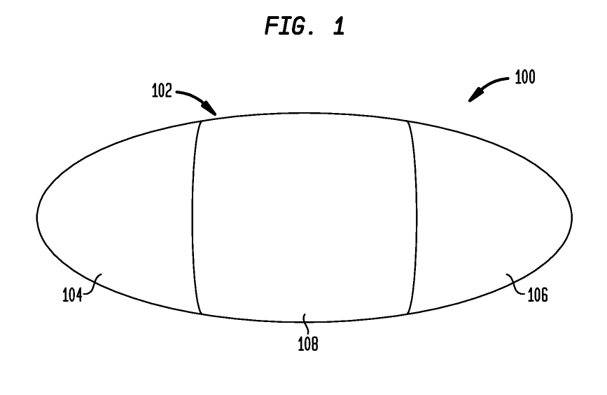

FIG. 1 illustrates an exemplary embodiment of an electrode device 100 in the form of a swallowable capsule. As shown, device 100 includes an outer housing 102 having an anode 104 and cathode 106 preferably positioned on opposite sides of the outer surface of housing 102 with an insulating portion 108 therebetween. Housing 102 may contain the necessary electrical components (not shown) for applying an electrical impulse to anode 104 and cathode 106. Alternatively, electrodes 104, 106 may be located within housing 102 and housing 102 may have a conductive outer surface such that electrical impulses can be passed from electrodes 104, 106 through the outer surface of housing to a target region within the patient. This would prevent direct contact between the electrodes 104, 106 and the intestinal walls.

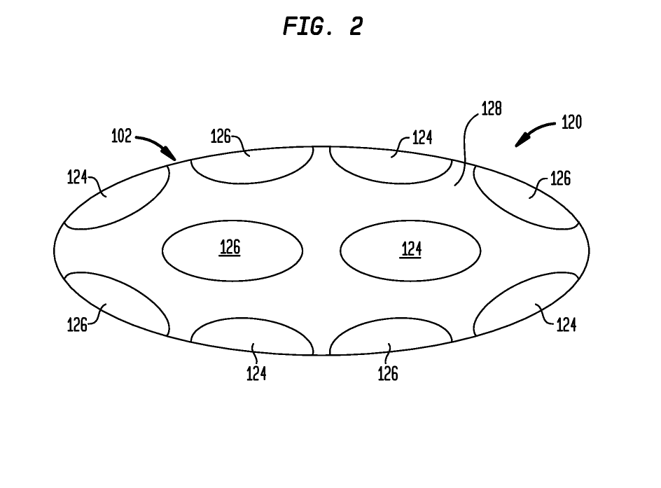

FIG. 2 illustrates another alternative embodiment of the present invention. As shown, electrode device 120 includes an outer housing 122 having a plurality of cathodes 124 and anodes 126 arranged in an alternating checkerboard pattern on the outer surface of housing 122. Housing 122 comprises an insulating surface 128 between anodes 126 and cathodes 124. A power source (not shown) delivers an appropriate electrical impulse wherever opposite polarity electrodes came in contact with the walls of the intestines.

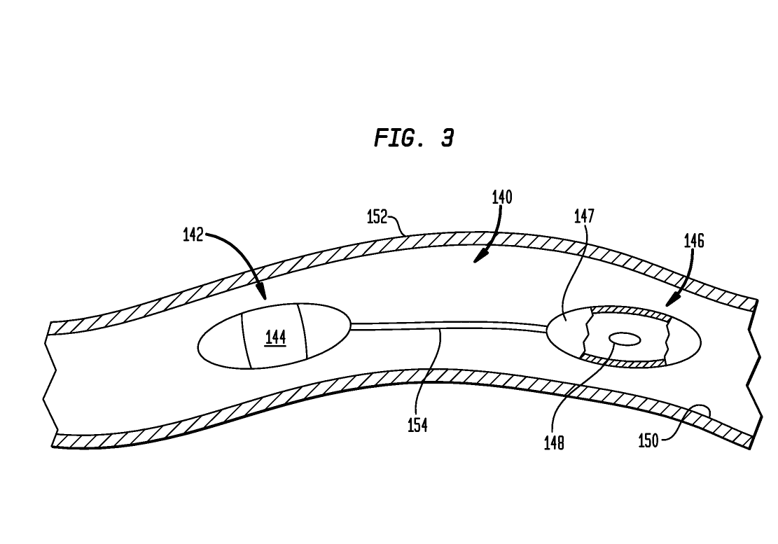

FIG. 3 illustrates yet another alternative embodiment of the present invention. In this embodiment, the opposite polarity electrodes (i.e., anode and cathode) are contained in separate housings. As shown, electrode device 140 includes a first housing 142 having a cathode 144 on the outer surface of first housing 142. Alternatively, cathode 144 may be located within housing 142 as discussed above. In addition, the entire outer surface of housing 142 may be electrified so that it does not matter which portion of housing 142 contacts inner wall 150 of intestines 152. Device 140 further includes a second housing 146 with a conductive outer surface 147 and an anode 148 within outer surface 147. Surface 147 is preferably a thin porous covering that allows for electrical impulses to pass therethrough and to avoid accidental direct contact with cathode 144, preventing short circuiting and preventing anodic contact with the walls 150 of the intestines 152. As shown, first and second housings 142, 146 are couple to each other with a small wire attachment 154, allowing anode 148 to trail behind cathode 144 as the device 140 passes through the intestines 152. Wire 154 may be semi-rigid to ensure that anode 148 and cathode 144 are separated by a selected distance, if necessary.

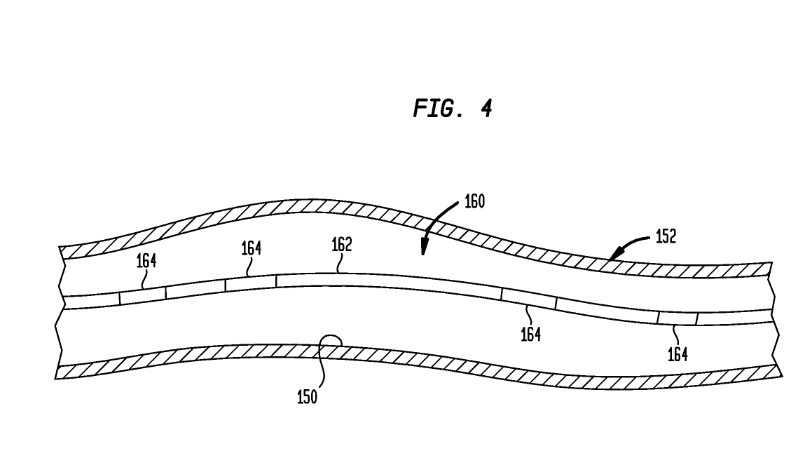

Referring now to FIG. 4, an electrode device 160 includes a long thin cable 162 comprising a series of nodes or electrodes 164 that are configured to pulse in sequence to pace the stomach and/or intestines 152 into proper function. Cable 162 is advanced through the esophagus (not shown) and into the target position within the stomach and/or intestines 152. Cable 162 has a width of about 1-4 mm, preferably about 2.5 mm, and electrodes 164 are about 2-7 mm wide, preferably about 5 mm and about 0.2 to 3 cm long, preferably about 1 cm. Cable 162 may be driven externally through the gastrointestinal tract until its distal end has reached the rectum. At this point, the proximal end is disconnected from the signal generator and the device is pulled free.

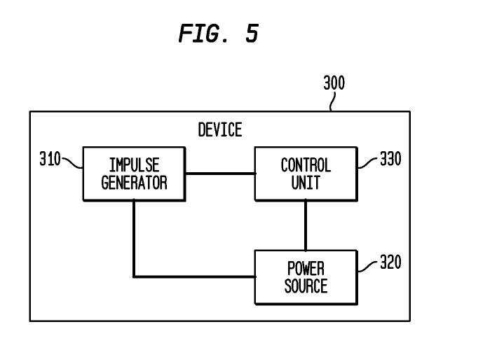

FIG. 5 is a schematic diagram of a nerve modulating device 300 for delivering electrical impulses to nerves according to one embodiment of the present invention. As shown, device 300 may include an electrical impulse generator 310; a power source 320 coupled to the electrical impulse generator 310 and a control unit 330 in communication with the electrical impulse generator 310 and coupled to the power source 320. Generator 310 is coupled to an electrode device 100 (not shown in FIG. 5) remotely through a wireless transmitter and receiver. The control unit 330 may control the electrical impulse generator 310 for generation of a signal suitable for amelioration of a patient's condition when the signal is applied via the electrode device 100 to the target region of the patient. It is noted that nerve modulating device 300 may be referred to by its function as a pulse generator. U.S. Patent Application Publications 2005/0075701 and 2005/0075702, both to Shafer, both of which are incorporated herein by reference, relating to stimulation of neurons of the sympathetic nervous system to attenuate an immune response, contain descriptions of pulse generators that may be applicable to the present invention.

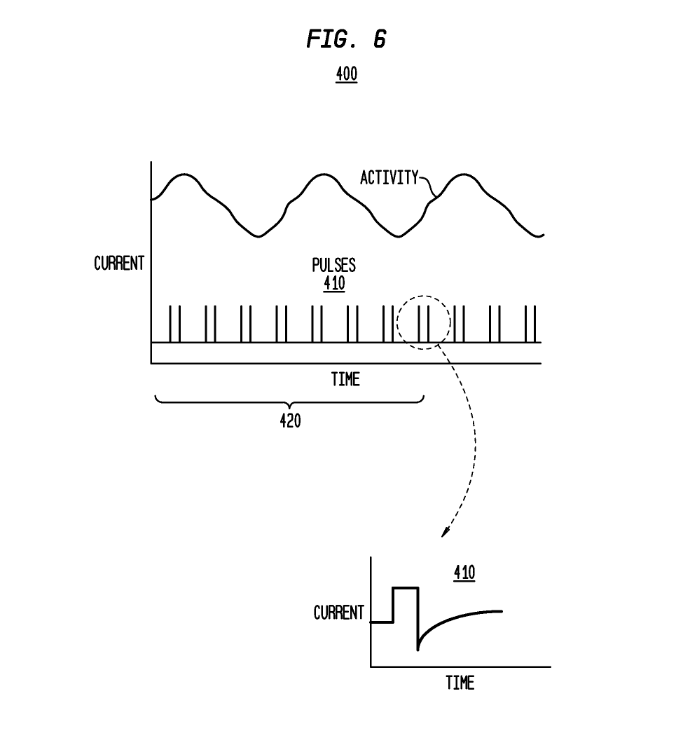

FIG. 6 illustrates an exemplary electrical voltage/current profile for a blocking and/or modulating inhibitory nerve signals in the intestines. Application of a suitable electrical voltage/current profile 400 may be achieved using the pulse generator 310. In a preferred embodiment, the pulse generator 310 may be implemented using the power source 320 and the control unit 330 having, for instance, a processor, a clock, a memory, etc., to produce a pulse train 420 to the electrode(s) that deliver the impulses 410 to the patient's intestines.

In the preferred embodiment, the parameters of the drive signal are designed to emulate the action potential spikes of the intestines. The most frequent type of movement of the small intestine is called segmentation. Segmentation is characterized by closely spaced contractions of the circular muscle layer. These contractions divide the intestines into small neighboring segments. In rhythmic segmentation, the sites of the circular contractions alternative so that an individual segment of gut contracts and then relaxes. In contrast to segmentation, peristalsis is the progressive contraction of successive sections of circular smooth muscles. The contractions move along the gastrointestinal tract in an orthograde direction. The slow waves of the smooth muscle cells determine the timing of intestinal contractions.

The typical frequency of regular slow waves in humans is highest in the duodenum (about 11 to 13 per minute) but declines along the length of the small intestine to a minimum of about 8 or 9 per minute. The slow waves are typically accompanied by bursts of action potential spikes. When action potentials occur, they elicit strong smooth muscle contractions that cause the major mixing and propulsive movements of the small intestines. Thus, in the preferred embodiments of the present invention, the drive signal shall provide rapid spikes of electrical pulses occurring at the peaks of the slow waves or depolarizations, typically between about 8 to 15 per minute. The rapid spikes are preferably in a frequency range substantially corresponding to the frequencies of the action potential spikes in the human intestines. In the exemplary embodiment, these rapid spikes will be about 1 to 20 Hz, preferably about 1-5 Hz. The drive signal may have a peak voltage amplitude selected to influence the therapeutic result, such as about 0.2 volts or greater, such as about 0.2 volts to about 20 volts. The electric or electromagnetic field may be administered for a predetermined duration, such as between about 5 minutes and about 1 hour, or between about 5 minutes and about 24 hours.

The protocol of one or more embodiments of the present invention may include measuring a response of the patient to the applied current and/or field(s). For example, the digestive muscle activity of the patient may be monitored and the parameters of the drive signal (and thus the induced current and/or fields) may be adjusted to improve the treatment.

Among the available devices to implement the control unit and/or signal generator for facilitating the induced current and/or the emission of electric fields and/or electromagnetic fields is a physician programmer, such as a Model 7432 also available from Medtronic, Inc. An alternative control unit, signal generator is disclosed in U.S. Patent Publication No.: 2005/0216062, the entire disclosure of which is incorporated herein by reference. U.S. Patent Publication No.: 2005/0216062 discloses a multi-functional electrical stimulation (ES) system adapted to yield output signals for effecting faradic, electromagnetic or other forms of electrical stimulation for a broad spectrum of different biological and biomedical applications. The system includes an ES signal stage having a selector coupled to a plurality of different signal generators, each producing a signal having a distinct shape such as a sine, a square or saw-tooth wave, or simple or complex pulse, the parameters of which are adjustable in regard to amplitude, duration, repetition rate and other variables. The signal from the selected generator in the ES stage is fed to at least one output stage where it is processed to produce a high or low voltage or current output of a desired polarity whereby the output stage is capable of yielding an electrical stimulation signal appropriate for its intended application. Also included in the system is a measuring stage which measures and displays the electrical stimulation signal operating on the substance being treated as well as the outputs of various sensors which sense conditions prevailing in this substance whereby the user of the system can manually adjust it or have it automatically adjusted by feedback to provide an electrical stimulation signal of whatever type he wishes and the user can then observe the effect of this signal on a substance being treated. It is noted that if the aforementioned hardware requires modification to achieve the parameters of the drive signals, then one skilled in the art would not require undue experimentation to achieve such modifications, or one skilled in the art would readily be able to obtain hardware capable of producing the drive signals based on the description herein.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating intestinal peristalsis in a patient, comprising:
introducing an electrode through the esophagus and into the digestive tract of the patient; and
applying an electrical impulse to the electrode to modulate one or more nerves within the digestive tract such that intestinal peristalsis function is at least partially improved.

2. The method of claim 1 wherein the introducing step is carried out by positioning the electrode within the patient's mouth such that the electrode can be swallowed by the patient.

3. The method of claim 1 wherein the introducing step comprises advancing the electrode through the esophagus and into the small intestines of the patient with an introducing device.

4. The method of claim 1 wherein the electrical impulse is sufficient to initiate peristalsis in a patient suffering from a temporary arrest of peristalsis.

5. The method of claim 1 further comprising: advancing the electrode through the digestive tract until the electrode reaches a target region with the digestive tract wherein peristalsis is arrested; and
applying an electrical impulse to the electrode after the allowing step to initiate peristalsis at the target region of the digestive tract.

6. The method of claim 5 further comprising advancing the electrode through the digestive tract distal to the target region after the applying step.

7. The method of claim 5 wherein the advancing step is carried out by allowing the electrode to advance through the digestive tract through natural peristalsis.

8. The method of claim 5 wherein the advancing step is carried out by applying a magnetic field to the electrode.

9. The method of claim 8 further comprising:
holding the electrode at the target region with a magnet during the applying step; and
advancing the electrode distal to the target position with the magnet after peristalsis has at least partially improved at the target region.

10. The method of claim 5 wherein the electrode is coupled to a flexible cord extending from an exterior portion of the patient to the electrode, wherein the electrode is advanced by advancing the flexible cord.

11. The method of claim 1 wherein the electrode comprises a cathode and an anode.

12. The method of claim 1 wherein the electrode is an active electrode, the method further comprising introducing a return electrode through the esophagus and into the digestive tract of the patient and applying an electrical signal between the active and return electrodes.

13. The method of claim 12 wherein the active and return electrodes are coupled to each other.

14. The method of claim 1 wherein the electrical impulse has a frequency of about 1 to 20 Hz.

15. The method of claim 14 wherein the frequency is between about 1 Hz to 5 Hz.

16. The method of claim 14 wherein about 1-10 electrical impulses are delivered about 8 to 15 times per minute.

17. The method of claim 1 wherein the electrical impulse has a peak amplitude of about 10 to about 20 volts.

18. The method of claim 1 wherein the electrical impulse has a pulse width of about 100 us to about 300 us.

19. A device for treating intestinal peristalsis comprising:
an electrode configured for introduction through an esophagus of the patient and being advanced through a digestive tract of the patient; and
a source of electrical energy electrically coupled to the electrode and configured to apply an electrical impulse to the electrode sufficient to at least partially improve intestinal peristalsis function of the patient.

20. The device of claim 19 wherein the electrode is a pill sized for advancement through the esophagus and into the digestive tract.

21. The device of claim 19 further comprising an introducer configured for advancing the electrode through the esophagus of the patient to a target region within the digestive tract.

22. The device of claim 21 wherein the introducer comprises a scope.

23. The device of claim 19 wherein the electrode comprises a cathode and an anode.

24. The device of claim 19 wherein the electrode is an active electrode, the device further comprising a return electrode configured for advancement through the esophagus and into the digestive tract.

25. The device of claim 24 wherein the return electrode is coupled to the active electrode.

26. The device of 19 further comprising a flexible cord coupled to the electrode and configured for advancement through the esophagus and the digestive tract.

27. The device of 23 wherein the cathode is on an opposite side of the electrode from the anode.

28. The device of claim 19 wherein the electrode comprises multiple anodes and cathodes.

29. The device of claim 24 wherein further comprising a porous housing surrounding the return electrode and configured to minimize contact between the return and active electrodes.

30. The device of claim 19 further comprising:
a flexible cable configured for advancement through the esophagus and the digestive tract of a patient; and
a plurality of electrodes coupled to the cable and spaced from each other.

31. The device of claim 30 wherein the source of electrical energy is coupled to the plurality of electrodes and configured to deliver electrical signals such that the electrodes pulse in sequence to pace the digestive tract.
